(19) United States
(12) Patent Application Publication
(10) Pub. No.: US 2005/0244801 A1
(43) Pub. Date: Nov. 3, 2005

(54) System and method of training the proper placement of airway adjuncts in a training manikin

(76) Inventor: Antonino DeSalvo, Brooklyn, NY (US)

Correspondence Address: HOWARD C. MISKIN;C/O STOLL, MISKIN, & BADIE, THE EMPIRE STATE BUILDING, 350 FIFTH AVENUE SUITE 4710, NEW YORK, NY, 10118, US

(21) Appl. No.: 10/833,765

(22) Filed: Apr. 28, 2004

(51) Int. Cl. G09B 23/28; A61B 1/267

(52) U.S. Cl. 434/262; 600/300

(57) ABSTRACT

A system and method of training the proper placement of an endotracheal tube in a training manikin's trachea using a live video camera located inside the manikin for simultaneous remote display of the manikin's trachea and surrounding area and the relative positioning of the laryngoscope blade and endotracheal tube. The system includes a training manikin, a micro video camera mounted adjacent the nasal cavity of the manikin, with the camera directed at the trachea, a monitor displaying the images being captured by the video camera, and electrical connection connecting the video camera to said monitor.

### FIELD OF THE INVENTION

The invention relates to a system and method of training personnel in the proper placement of airway adjuncts in a training manikin. In particular, a system and method of training the proper placement of an endotracheal tube in a training manikin's larynx by using a live video camera located inside the manikin for simultaneous remote display of the manikin's larynx and surrounding area and the relative positioning of the laryngoscope blade and endotracheal tube.

### BACKGROUND OF THE INVENTION

The proper and prompt placement of an endotracheal tube in a person's trachea, i.e. intubation, is important in a life saving situation. Therefore, it is necessary for emergency paramedics, other health care professionals and others that may require this skill, to develop the skill of properly and promptly placing an endotracheal tube in a person's trachea, which is below the larynx. Such skill is generally taught in a clinical classroom setting with a training manikin having the required anatomical structures that resemble a human's upper body, including the respiratory system. Typically, a student peers over the shoulder of the instructor trying to see what the instructor sees as the laryngoscope is placed in the throat to expose the larynx and vocal cords for insertion of an endotracheal tube. The student cannot obtain a proper view since he/she has a different perspective than the instructor, which hinders the student's ability to appreciate what the instructor is demonstrating. Similarly, when a student practices the insertion of a laryngoscope into a training manikin, the instructor must try to see over the student's shoulder to provide instant feedback and an evaluation of the student's performance. However, the instructor's view is hindered and cannot provide efficient feedback and proper evaluation.

An attempt to provide instantaneous evaluation and feedback of practicing the intubation technique is U.S. Pat. No. 6,123,666 to K. D. Wrenn et al. The '666 patent discloses a laryngoscope blade having an integral fiber optic scope mounted adjacent the end of the blade to enable remote observation of the airway and surrounding area during insertion of the laryngoscope blade. Fiber optics for illumination and viewing are provided. Although the laryngoscope blade disclosed in the '666 patent provides remote observation of the intubation technique during teaching and evaluation, it disadvantageously requires the laryngoscope to be connected to an external source via a tubular conduit 40, which is cumbersome and interferes with experiencing the use of a regular laryngoscope in a real life situation. Further, if the tubular conduit 40 becomes entangled while being used in a real life situation, it may cause unnecessary interference with the performance of the intubation technique. Due to the positioning of the fiber optic scope adjacent the end of the laryngoscope blade, the device disclosed in the '666 patent also disadvantageously restrict viewing to only during the insertion of the laryngoscope blade. Once the laryngoscope blade is inserted into the larynx, it remains in that position while an endotracheal tube is fed into the trachea. Therefore, a user can only blindly insert the endotracheal tube into the larynx with the scope directed in a forward position into the trachea. A user of the device disclosed in the '666 patent will not have any reference point for the proper placement of the endotracheal tube.

Therefore, there is a need for a system and method of training personnel in the proper placement of an endotracheal tube that does not interfere with the use of a laryngoscope and provides improved views of both the laryngoscope and endotracheal tube during the intubation procedure.

### SUMMARY OF THE INVENTION

The present invention provides a system and method of training personnel in the proper placement of an endotracheal tube in a training manikin's trachea using a live video capturing device located inside the manikin for simultaneous remote display of the manikin's larynx and the relative positioning of the laryngoscope blade and endotracheal tube.

The system of the present invention comprises a training manikin, a micro video camera mounted adjacent the nasal cavity inside the manikin directed to view distal to the skull, at the larynx, a remote monitor and an electrical connection connecting the video camera to the remote monitor.

The method of the present invention comprises the steps of providing a training manikin, providing a micro video camera adjacent the nasal cavity inside the manikin directed to view distal to the skull, at the larynx, inserting the larynogoscope blade adjacent the larynx, inserting an endotracheal tube below the larynx, providing a remote monitor displaying the larynogoscope and endotracheal tube being inserted adjacent and below the larynx, respectively, and providing an electrical connection connecting the video camera to the remote monitor.

### BRIEF DESCRIPTION OF THE DRAWINGS

A preferred embodiment of the present invention has been chosen for purposes of illustration and description and is shown in the accompanying drawings forming a part of the specification wherein:

FIG. 1 is a perspective view of the system of the present invention.

FIG. 2 illustrates the anatomical area where an endotracheal tube is placed and the view from a remote monitor.

FIG. 3 illustrates the placement of the laryngoscope blade tip and the view from a remote monitor.

### DETAILED DESCRIPTION OF THE PREFERRED EMBODIMENTS

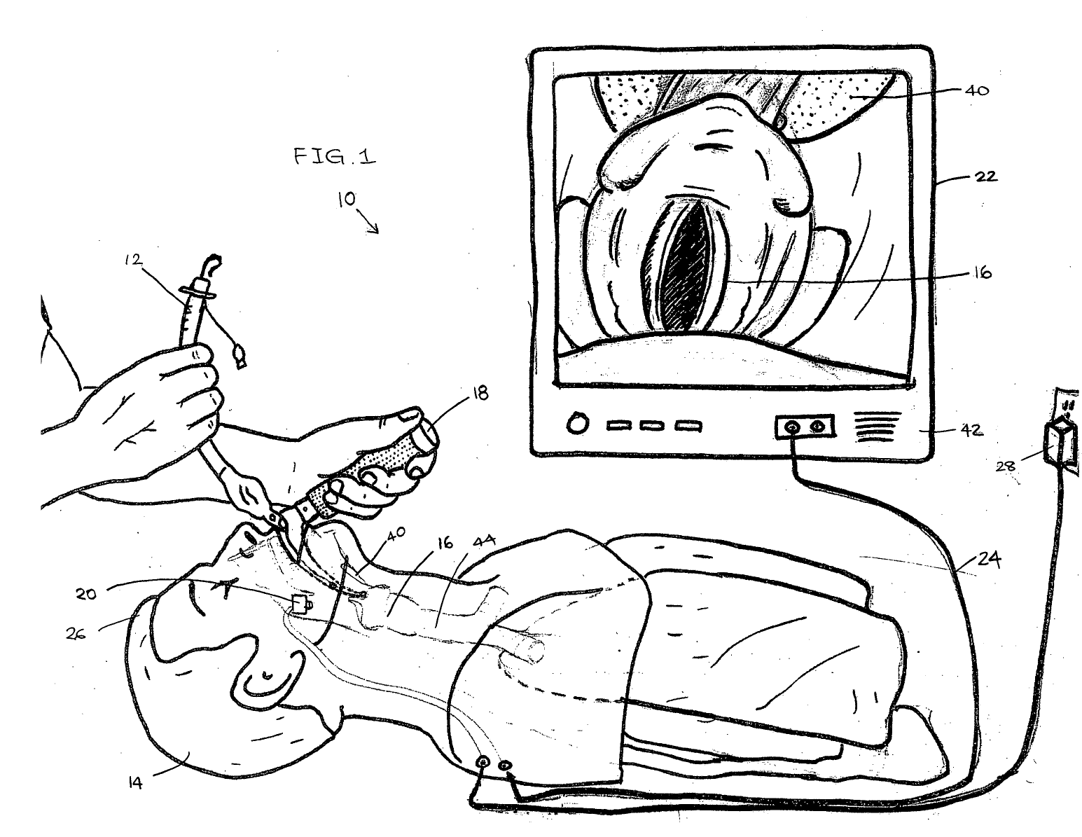

With reference to the drawings, wherein the same reference number indicates the same element throughout, there is shown in FIG. 1 a system 10 of training the proper placement of an endotracheal tube 12 in a training manikin's 14 larynx 16 using a laryngoscope 18, comprises a training manikin 14, a micro video camera 20, a remote monitor 22 and an electrical connection 24 connecting the video camera 20 to the monitor 22.

Training manikin 14 can be any airway training manikin that provides the necessary anatomical features for training the insertion of endotracheal tube 12. One such training manikin is the Laerdal™ Airway Management Trainer, which provides a lifelike upper torso and head that simulates real-world complications when practicing intubation technique.

A micro video camera 20 is mounted in the nasal cavity inside the manikin 14 directed to view distal to the skull 26, at the larynx 16. Alternatively, the video camera 20 may be mounted at another location, such as on the roof of the mouth, so long as it is directed to view distal to the skull 26, at the larynx 16. A micro video camera 20 is used to allow its placement in the rather limited internal space of the manikin 14. Micro video cameras 20 generally available at spy shops are coin sized and can be used for the present invention. Power supply to the video camera 20 is provided either from an external power source such as an AC adaptor 28 or batteries (not shown).

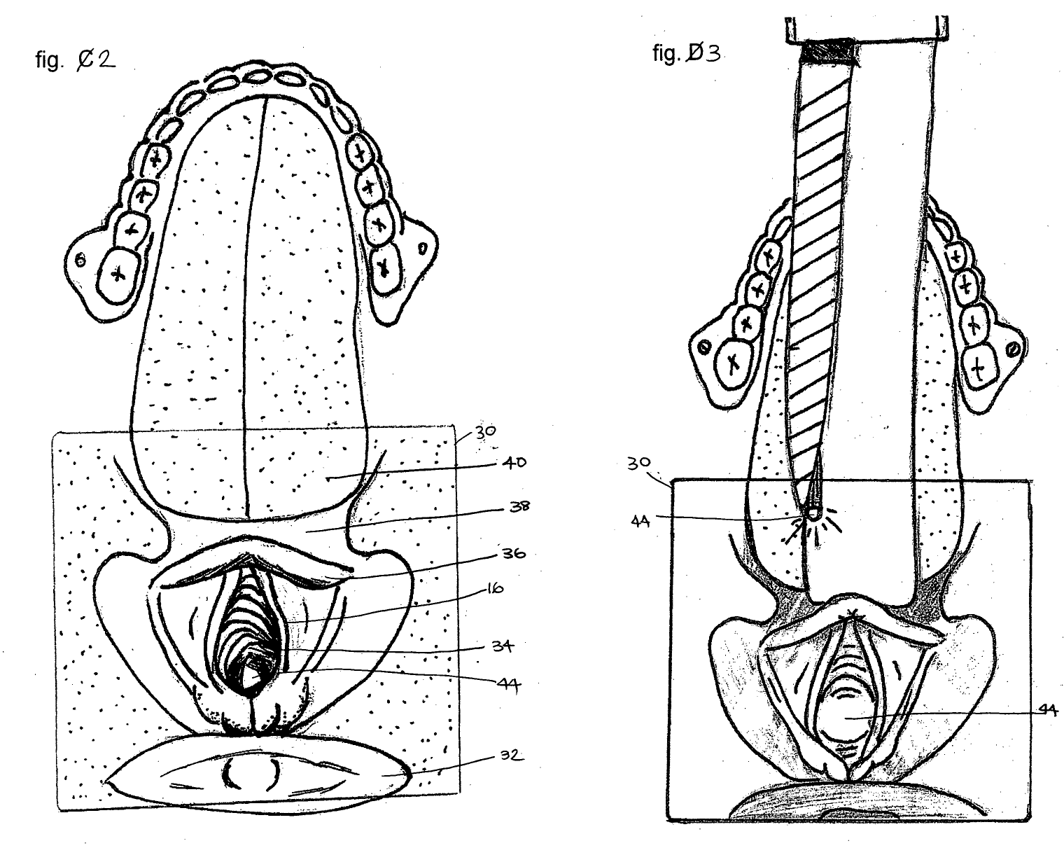

A remote monitor 22 such as a television, a laptop monitor, etc. can be used to display the images collected by the video camera 20. The video camera 20 is connected to the remote monitor 22 via electrical connection 26 such as A/V (audio/video) cables, USB connection, wireless transmission, etc. The rectangular box 30 in the lower portion of FIG. 2 illustrates the view of the internal organs of the manikin 14 as seen on the monitor 22. As shown in FIG. 2, the anatomy surrounding the larynx 16 are shown: esophagus 32, vocal cord 34, epiglottis 26, vallecula space 38 and tongue 40. A recording device 42, such as a VCR recorder, CD-ROM burner or DVD burner, may be provided to record the images for later review and/or evaluation.

With the system 10, an instructor inserts the blade 18a of the larynogoscope 18 into the larynx area 16 of the manikin 14 while the students observe via the remote monitor 22. The larynogoscope 18 may have a small light source 44, such as an LED, attached adjacent to the end of the blade 18a to illuminate the surrounding area during insertion of the larynogoscope 18. The instructor can point to certain anatomical landmarks in the areas surrounding the larynx 16 prior to the insertion of the blade 18a and can also demonstrate improper insertion of the blade 18a for teaching purposes. Once the blade 18a of the larynogoscope 18 is in the proper location in the vallecula space 28 adjacent the epiglottis 36, as shown in FIG. 3 (rectangular box 30 at the lower portion of FIG. 3 illustrates the view as seen on the monitor 22), the endotracheal tube 12 can then be inserted into position below the larynx 16 at the trachea 44. The entire intubation procedure, with unobstructed views, is available to the students and/or recorded for later review and analysis. This advantageously enhances the teaching of the proper placement of airway adjuncts in a training manikin 14.

Similarly, with the system 10, when a student performs the intubation procedure, an instructor can view the monitor 22 and provide instant evaluation and feedback to the student as to the placement of the blade 18a of the laryngoscope 18 and the endotracheal tube 12. Also, the entire intubation procedure may be recorded for later review and/or analysis by an instructor. This advantageously improves the teaching technique of the intubation procedure.

The features of the invention illustrated and described herein is the preferred embodiment. Therefore, it is understood that the appended claims are intended to cover the variations disclosed and unforeseeable embodiments with insubstantial differences that are within the spirit of the claims.

1. A system for training the proper placement of an endotracheal tube in a trachea of a training manikin having a nasal cavity, comprises:
a. a training manikin,
b. a micro video camera being mounted adjacent the nasal cavity and inside said manikin, wherein said camera is directed at the trachea,
c. a monitor displaying the images being captured by said video camera, and
d. means for electrically connecting said video camera to said monitor.

2. The system of claim 1 wherein said manikin is an airway training manikin.

3. The system of claim 1 wherein said training manikin having a mouth, said video camera being mounted on the roof of the mouth.

4. The system of claim 1 wherein said video camera is coin sized.

5. The system of claim 1 further comprises an external power source for said video camera.

6. The system of claim 1 wherein said monitor is a television.

7. The system of claim 1 wherein said monitor is a laptop monitor.

8. The system of claim 1 wherein said monitor is located at a remote location.

9. The system of claim 1 wherein said electrically connecting means comprises A/V cables.

10. The system of claim 1 wherein said electrically connecting means comprises a USB cable.

11. The system of claim 1 wherein said electrically connecting means comprises a wireless connection.

12. The system of claim 1 further comprises means for recording the images being captured by said video camera

13. The system of claim 12 wherein said recording means comprises a VCR recorder.

14. The system of claim 12 wherein said recording means comprises a CD-ROM burner.

15. The system of claim 12 wherein said recording means comprises a DVD burner.

16. The method of training the proper placement of an endotracheal tube in a trachea of a training manikin having a nasal cavity, using a larynogoscope having a blade, comprises the steps of:
a. providing a training manikin,
c. providing a micro video camera adjacent the nasal cavity and inside said manikin, wherein said video camera is directed at the trachea,
d. inserting the blade of the larynogoscope above the trachea,
e. inserting an endotracheal tube into the trachea,
f. providing a monitor displaying the larynogoscope and endotracheal tube being inserted above and into the trachea, respectively, as captured by said video camera,
g. providing means for electrically connecting said video camera to said monitor.

17. The method of claim 16 wherein said training manikin is an airway training manikin.

18. The method of claim 16 wherein said training manikin having a mouth, said video camera being mounted on the roof of the mouth.

19. The method of claim 16 wherein said video camera is coin sized.

20. The method of claim 16 further comprises the step of providing an external power source for said video camera.

21. The method of claim 16 wherein said monitor is a television.

22. The method of claim 16 wherein said monitor is a laptop monitor.

23. The method of claim 16 wherein said monitor is located at a remote location.

24. The method of claim 16 wherein said electrically connecting means comprises A/V cables.

25. The method of claim 16 wherein said electrically connecting means comprises a USB cable.

26. The method of claim 16 wherein said electrically connecting means comprises a wireless connection.

27. The method of claim 16 further comprises the step of providing means for recording the larynogoscope and endotracheal tube being inserted above and into the trachea, respectively, as captured by said video camera.

28. The method of claim 27 wherein said recording means comprises a VCR recorder.

29. The method of claim 27 wherein said recording means comprises a CD-ROM burner.

30. The method of claim 27 wherein said recording means comprises a DVD burner.
